(19) United States
(12) Patent Application Publication
(10) Pub. No.: US 2017/0241905 A1
(43) Pub. Date: Aug. 24, 2017

(54) Advanced Aircraft Fuel Tank And Water Detection Device

(71) Applicant: Zodiac Aerotechnics, Roche la Moliere (FR)

(72) Inventor: Bruno Reynard, Francheville (FR)

(21) Appl. No.: 15/436,133

(22) Filed: Feb. 17, 2017

(30) Foreign Application Priority Data
Feb. 22, 2016 (FR) 1651410

(51) Int. Cl. G01N 21/41; B64D 37/02; G01N 33/28

(52) U.S. Cl. CPC G01N 21/4133; G01N 33/2847; B64D 37/02

(57) ABSTRACT

An aircraft fuel tank, which includes a water sensor, arranged at the bottom of the tank and connected to a calculation and processing mechanism in order to receive a presence of water signal sent by the sensor, and to return information regarding the need, or not, to perform a fuel tank drainage operation.

### FIELD OF THE INVENTION

This invention relates to the art of aircraft and concerns an advanced fuel tank for aircraft, especially for airplanes, helicopters, or similar, as well as a device for the detection of water, particularly at the bottom of such a fuel tank.

### BACKGROUND OF THE INVENTION

In the art of aeronautics, it is relatively common to have water accumulating at the bottom of aircraft fuel tanks. This water enters the tank in the form of water dissolved in the fuel, or else it is related to the condensation of the air within the tank and which enters therein when the aircraft changes altitude, or else it is related to maintenance operations, or else has any other origin. The presence of this water requires drainage operations of said tanks.

Tank drainage operations are performed manually in accordance with the recommendations of the aircraft manufacturer, at a predetermined frequency, usually at regular intervals in time of a few flights or a few days. Drainage operations have the drawback of leading to relatively long aircraft immobilization time, insofar as before operating the drainage system, once the aircraft has landed, it is necessary for it to thaw.

Given that these operations are performed at predetermined intervals, unnecessary drainage operations are thus occasionally performed when there is no water at the bottom of the tanks or when the amount of water is relatively low, thereby leading to aircraft immobilization and unnecessary expenses.

The current state of the art provides no solution to these drawbacks.

### SUMMARY OF THE INVENTION

One of the objects of the invention is therefore to remedy these drawbacks by proposing an aircraft fuel tank that makes it possible to avoid aircraft immobilization and unnecessary expenses relating to drainage operations of said tank.

To this purpose, an aircraft fuel tank has been developed which is remarkable in that it includes a water sensor, arranged at the bottom of said tank and connected to calculation and processing means in order to receive a presence of water signal sent by said sensor, and to return information regarding the need, or not, to perform a fuel tank drainage operation.

In this way, calculation and processing means are able to indicate, based upon a signal sent by the sensor, if it is necessary or not to perform a tank drainage operation. Unnecessary drainage operations are therefore avoided, thus limiting aircraft immobilization and unnecessary expenses.

According to a specific embodiment, the sensor comprises an optical component connected to calculation and processing means by means of at least one optical fiber, said optical component being intended to make it possible to differentiate between water and fuel.

In this way, the detection of water is based upon an optical phenomenon that is stable over time and that makes it possible to avoid any calculation method that would consider the level of fuel present within the tank in order to measure the amount of residual water. The implementation of at least one optical fiber is advantageous insofar as it makes it possible to avoid the presence of other materials, such as copper. The safety of the system is thus guaranteed.

According to a specific embodiment, the optical fiber is connected, on the one hand, to the optical component and, on the other hand, to a light source in order to send at least one light beam to said optical component, said optical component being suitable:
to reflect and return the light beam within the optical fiber to the calculation and processing means when said optical component is immersed in water, and;
to refract and not return the light beam when said optical component is immersed in fuel.

The invention is particularly advantageous insofar as it makes use of the physical properties of water and fuel such as the refractive index, which is stable over time, in order to differentiate between water and fuel. No moving parts are implemented such that the maintenance of the sensor is limited, or even non-existent.

According to a specific embodiment, the optical component is in the form of a prism with an isosceles triangular section in such a manner as to define at least one main side and two converging sides. The prism is made of a material that is transparent to light with a refractive index equal to n1, wherein n1 is greater than 1.33. The optical fiber is connected orthogonally to the main side of the prism such that the incident light beam is intended to refract and/or reflect on a main convergent side with an angle i1 relative to the normal of the first convergent side. The prism is designed such that: n1×sin(i1)>1.33 which is the value of the refractive index of water.

The characteristics of the prism are calculated by applying Snell-Descartes law which indicates that: n1×sin(i1)=n2×sin(i2), wherein n2 corresponds to the refractive index of the medium within which the prism is immersed, and i2 corresponds to the angle of the refracted beam relative to the normal of the first convergent side of the prism.

In this way, in order to detect the presence of water at the bottom of the fuel tank, it is necessary for the incident beam to be totally reflected by the prism when the latter is immersed in water. In order to do this, it is necessary to calculate the refraction limit of the incident beam.

Given that water has a lower refractive index than that of the prism, the more the angle of incidence of the light beam increases, the more the beam refracted within water deviates from the normal to the convergent side. Thus, the refraction limit is reached when the refracted beam touches the surface of the first convergent side of the prism, i.e., when the angle i2 is equal to 90° relative to the normal of the first convergent side of the prism.

Thus, by applying Snell-Descartes law:

n1×sin(i1)=n2×sin(i2)

n1×sin(i1)=1.33×sin(90°)

n1×sin(i1)=1.33

When the prism is designed such that: n1×sin(i1)=1.33, the incident light beam is refracted at the refractive limit, it touches the first convergent side of the prism. This means that when the prism is designed such that: n1×sin(i1)>1.33 the incident beam is no longer refracted. The incident beam exceeds the refractive limit and is completely reflected by the first convergent side. The incident beam is then reflected onto the second convergent side and is sent to the calculation and processing means by means of the optical fiber, which makes it possible to detect the presence of water.

By construction, the convergence angle of the convergent sides of the prism is equal to the angle of incidence of the light beam relative to the normal of the first convergent side of the prism. Thus, in order to implement the invention, the convergence angle of the convergent sides should be adjusted relative to the main side of the prism, based upon the refractive index of said prism.

The principle of the invention consists in detecting water by means of the complete reflection of the incident beam. In practice, the connection between the optical fiber and the prism is never entirely orthogonal, such that the detection of water can be performed by detecting a maximum intensity of reflected light. A small part of the incident light beam can still be refracted within the water without harm to the invention and without departing from the scope of the invention. The water will still be detected.

The prism is preferably made of a material with a refractive index n1 of between 1.33 and 1.40. According to a specific embodiment, the prism is made of Polysulfone, the refractive index of which is approximately equal to 1.38. In this way, when the Polysulfone prism is immersed in water and in applying the Snell-Descartes formula, a refractive limit is found for an incident angle equal to 74.5°:

n1×sin(i1)=1.33×sin(90°)

1.38×sin(i1)=1.33

sin(i1)=0964

i1=74.5°

This means that when the Polysulfone prism is immersed in water and the angle of the incident beam relative to the normal of the first convergent side is greater than 74.5°, the incident beam is no longer refracted. The incident beam is completely reflected and water is detected.

Preferably, the angle of the incident beam relative to the normal of the first convergent side is less than 87.5°, such that the incident beam is not fully reflected when the Polysulfone prism is immersed in fuel. Thus, the angle of the incident beam relative to the normal of the first convergent side is between 74.5° and 87.5°.

The fuel tank preferably comprises a plurality of sensors arranged at different levels from the bottom of the tank in order to know the amount of water present at the bottom of said tank, all of said sensors being connected to the same calculation and processing means.

The invention also concerns a tank comprising a drainage system arranged at the bottom of the tank and including, for example, a valve that can be actuated from the outside of the tank and one or several pipes for discharging the water. According to the invention, the water sensor is arranged within said drainage system.

The invention also relates to a water detection device, particularly within an aircraft fuel tank or within an aircraft fuel tank drainage system. Said device is remarkable in that it comprises at least one optical fiber connected, on the one hand, to an optical component and, on the other hand, to calculation and processing means and to a light source for sending at least one light beam to said optical component, said optical component being suitable:
to reflect and return the light beam within the optical fiber to the calculation and processing means when said optical component is immersed in water, and;
to refract and not return the light beam when said optical component is immersed in fuel.

Advantageously, the detection device according to the invention comprises a plurality of optical components connected by means of optical fibers to the same calculation and processing means and the same light source.

Thus, the invention makes it possible to multiplex the sensors, and to interrogate several sensors by means of a single calculation and processing means, thereby making it possible to reduce the weight, bulk and cost of the system to be integrated into an aircraft.

### BRIEF DESCRIPTION OF THE DRAWINGS

Further advantages and features will become more apparent from the following description, given by way of a non-limiting example, of a fuel tank according to the invention, from the attached drawings wherein:

FIG. 1 is a schematic view of a water sensor implemented within the fuel tank according to the invention;

FIG. 2 is a schematic view depicting a water detection system according to the invention, implementing the multiplexing of multiple sensors.

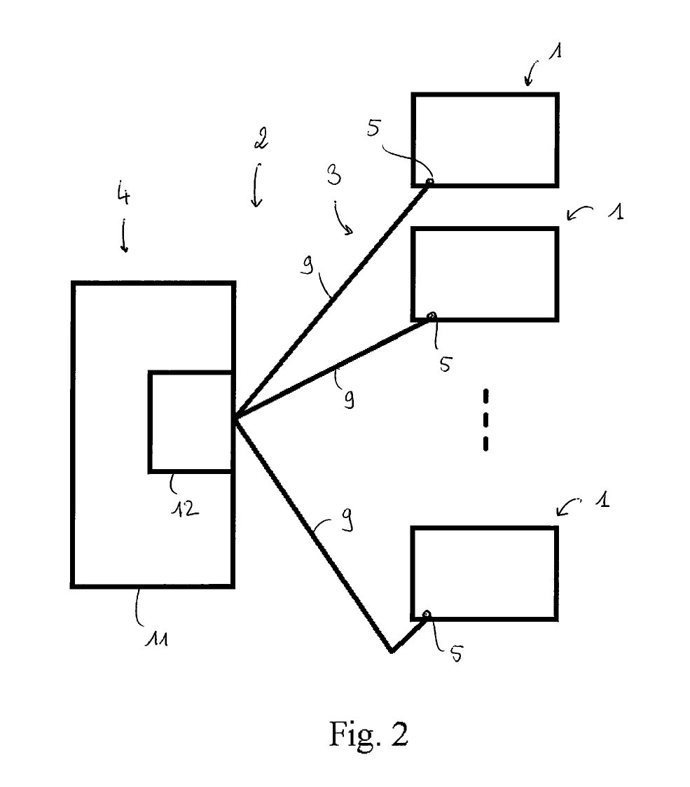

### DETAILED DESCRIPTION OF THE INVENTION

The invention relates to an aircraft fuel tank (1) comprising a water detection device (2) making it possible to provide information regarding the need, or not, to perform drainage operations in order to limit aircraft immobilization and unnecessary expenses.

More specifically, the detection device (2) comprises a water sensor (3), arranged at the bottom of said tank (1) and connected to calculation and processing means (4), for sending a presence of water signal.

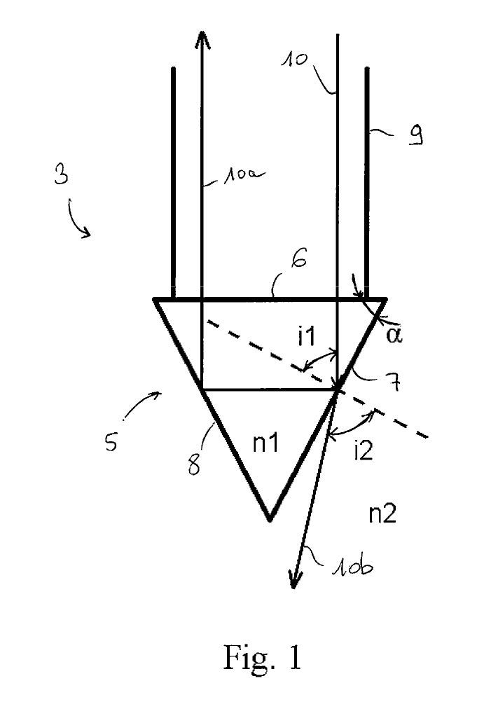

To this end, and with reference to FIG. 1, the water sensor (3) comprises a prism (5) with an isosceles triangular section. The prism (5) is for example made of Polysulfone and comprises a refractive index of 1.38. The prism (5) comprises a main side (6) and two convergent sides (7, 8) relative to the main side (6), at a convergence angle α.

An optical fiber (9) is connected, on the one hand, orthogonally to the main side (6) of the prism (5) and, on the other hand, to the calculating and processing means (4), which comprise a light source making it possible to send at least one light beam (10) through the prism (5) to the first convergent side (7).

Depending upon the medium within which the prism (5) is immersed, the light beam (10) is intended to refract, or completely reflect against the second convergent side (8) which therefrom returns the light beam (10) through the optical fiber (9) to the calculating and processing means (4).

The prism (5) is designed in such a way as to totally reflect the incident beam and return the reflected beam (10a) in the same direction as the incident beam (10) when immersed in water, and to transmit and refract the incident beam (10) when the prism (5) is immersed in fuel. The refracted beam is referenced as (10b) in FIG. 1.

Specifically, the convergence angle α of the convergent sides (7, 8) of the prism (5) is between 74.5° and 87.5°. By construction, the angle of incidence i1 of the light beam (10) relative to the normal of the first convergent side of the prism (5) is equal to the angle of convergence a and is therefore also between 74.5° and 87.5°. This angle corresponds to the refraction limit calculated according to Snell-Descartes law such that for a convergence angle α, and therefore an angle of incidence i1 of the light beam (10) of between 74.5° and 87.5°, the incident beam (10) is no longer refracted in water but is completely reflected to the second convergent side (8) and returned to the calculation and processing means (4) which thereby detect the presence of water, and the incident beam is always refracted within the fuel and never reflected.

The calculating and processing means (4) are of any suitable type in order to make it possible to receive a presence of water signal sent by said sensor (3), and to return information regarding the need, or not, to perform a fuel tank drainage operation. The calculation and processing means comprise for example a photo detector in order to determine when the light beam (10) is returned. The calculating and processing means (4) comprise a controller (11) and an interrogator (12) in order to interrogate the sensor (3) regarding the presence of water by means of the emission of a light beam (10). The calculating and processing means (4) are thereby suitable for interpreting said presence of water information in order to provide information regarding the need, or not, to perform a drainage operation based upon the amount of water present at the bottom of the tank (1). For example, a drainage operation can be recommended when the water exceeds a certain threshold.

Advantageously, the fuel tank (1) comprises a plurality of sensors (3) arranged at different levels from the bottom of tank (1). The various sensors (3) are all connected to the same calculation and processing means (4). Specifically, the optical fibers (9) make it possible to multiplex a plurality of prisms (5). The calculating and processing means (4) therefore make it possible to interrogate the different sensors (3) and according to their response information regarding the height of the water present within the tank (1) can be provided. In particular, this information makes it possible to calculate the amount of water present within the tank (1).

The prisms (5) can also be directly arranged at the bottom of the tank (1), for example within a drainage system that the tank (1) comprises. The drainage system arranged at the bottom of the tank comprises, for example, a valve that can be actuated from the outside of the tank and one or several pipes for discharging the water. The prisms (5) can be arranged within the valve or within the piping.

The water detecting devices (2) described above can be installed within all of the tanks (1) of the aircraft such that all of the prisms (5) are multiplexed and connected by means of optical fibers (9) to the same calculation and processing means (4), thereby making it possible to reduce the weight, bulk and cost of the system to be integrated into an aircraft.

It follows from the above that the tank (1) according to the invention comprises a water detection device (2) that makes it possible to provide information regarding the need to perform drainage operations in order to limit aircraft immobilization and unnecessary expenses.

What is claimed is:

1. An aircraft fuel tank comprising a water sensor, arranged at a bottom of said tank and connected to calculation and processing means in order to receive a presence of water signal sent by said sensor, and to return information regarding the need, or not, to perform a fuel tank drainage operation.

2. The fuel tank according to claim 1, characterized in that the sensor comprises an optical component connected to the calculation and processing means by means of at least one optical fiber, said optical component being intended to make it possible to differentiate between water and fuel.

3. The fuel tank according to claim 2, characterized in that the optical fiber is connected, on the one hand, to the optical component and, on the other hand, to a light source in order to send at least one light beam to said optical component, said optical component being suitable:
to reflect and return the light beam within the optical fiber to the calculation and processing means when said optical component is immersed in water, and;
to refract and not return the light beam when said optical component is immersed in fuel.

4. The fuel tank according to claim 3, characterized in that the optical component is in the form of a prism with an isosceles triangular section in such a manner as to define at least one main side and two convergent sides, said prism being made of a material that is transparent to light with a refractive index equal to n1, wherein n1 is greater than 1.33, the optical fiber is connected orthogonally to the main side of the prism such that the incident light beam is intended to refract, or both refract and reflect on a main convergent side with an angle i1 relative to the normal of the first convergent side, said prism being designed such that: n1×sin(i1)>1.33.

5. The fuel tank according to claim 4, characterized in that the refractive index of the prism n1 is less than 1.40.

6. The fuel tank according to claim 5, characterized in that the prism is made of Polysulfone and comprises a refractive index of 1.38, and wherein the convergence angle α between the convergent sides and the main side is between 74.5° and 87.5°.

7. The fuel tank according to claim 1, characterized in that said tank comprises a plurality of sensors arranged at different levels from the bottom of the tank in order to know the amount of water present at the bottom of said tank, all of said sensors being connected to the same calculation and processing means.

8. The fuel tank according to claim 1, characterized in that said tank comprises a drainage system arranged at the bottom of the tank, the water sensor being arranged within said drainage system.

9. A device for the detection of water, characterized in that said device comprises at least an optical fiber connected, on the one hand, to an optical component and, on the other hand, to calculation and processing means and to a light source for sending at least one light beam to said optical component, said optical component being suitable:
to reflect and return the light beam within the optical fiber to the calculation and processing means when said optical component is immersed in water, and;
to refract and not return the light beam when said optical component is immersed in fuel.

10. The water detection device according to claim 9, characterized in that said device comprises a plurality of optical components connected by means of optical fibers to the same calculation and processing means and the same light source.
